(19) United States
(12) Patent Application Publication
(10) Pub. No.: US 2011/0213224 A1
(43) Pub. Date: Sep. 1, 2011

(54) Back-aid skin inspection and complexion treatment device

(76) Inventor: Jack Merchant, Spokane, WA (US)

(21) Appl. No.: 12/932,550

(22) Filed: Feb. 28, 2011

Related U.S. Application Data
(60) Provisional application No. 61/339,320, filed on Mar. 1, 2010.

(51) Int. Cl. A61B 5/00; H04N 7/18

(52) U.S. Cl. 600/306; 348/77

(57) ABSTRACT

The invention provides a tool for improved self inspection of surface skin abnormalities of the human back. The Back-Aid device also enables one to treat complexion issues, precisely apply medicines or sunscreen, and monitor would healing by themselves. The device consists of a computer web-cam strategically mounted to a handle of rigid material which provides for easy manipulation over desired areas of the back. The images captured by the web-cam are displayed on a computer screen in which photo's and video's of images can be e-mailed to a doctor of the operators choice.

### CROSS REFERENCE TO RELATED APPLICATIONS

This application claims the benefit Of provisional patent application Ser. No. 61/339/320 filed 2010 Mar. 1 by the present inventor.

### BACKGROUND-PRIOR ART

The inventor has not found any Patents similar to this device.

Even with the best of mirrors it is still difficult to get the precision needed to do self skin inspection, treat complexion issues and apply medicine to most areas of the back. The Back Aid device has solved this visual problem allowing for precision self treatment complexion issues, skin conditions and inspection for skin abnormalities.

The world health organization states that nearly 66,000 people die yearly of skin cancer. About one American every hour dies in the United States of skin cancer. The key to surviving skin cancers, especially melanoma, is early detection and treatment. The American Dermatology association recommends regular self inspection of all areas of the body including the back for abnormalities. In the past hand held mirrors in front of a larger mounted mirror were the only means for self inspection of the human backside.

The back-Aid skin inspection device utilizes new use of a computer webcam mounted on a hand held plastic handle operating in conjunction with a computer to see a real time visual display of selected skin surfaces. The operator sits or stands in front of his computer and moves the device over his chosen areas of skin which is imaged by webcam to his computer display. The webcam is pointed toward the skin from the outside curved end of handle which rests against the skin to enable smooth movement of the device over skin surface areas he or she desires to see.

The operator can also attach complexion tools by way of a strap to the Back-Aid device and precisely treat blackheads/pimples on back with precision. The computer webcam can be lined up like the sights of a rifle with a selected complexion tool at the inside end of the handle. The wecam lenses are focused for clarity of skin surfaces and precise visual placement of the complexion tool. The operator can also apply medicines and creams to selected areas by cotton added to complexion tool.

Images and video's can be taken of either side of the human body and can and be stored to the computers hard drive from the webcam software and can be e-mailed to the doctor of their choice for follow-up if skin abnormalities are suspected.

### SUMMARY

In accordance of one embodiment of a hand held human skin inspection device for seeing selected areas either of the front or backside of the human body.

### ADVANTAGES

Accordingly several advantages of one or more aspects are as follows: to provide imaging and real time video of chosen skin areas of the human front or backside, the operator can inspect for skin abnormalities and compare images displayed at computer to images of known skin cancers, operators can apply medicines to areas of the backside as well as treat complexion issues by themselves, additionally operator can immediately e-mail any images obtained of concern to a doctor of his choice for follow-up.

### DRAWINGS-FIGURES

FIG. 1 is a an exploded side perspective view of a first embodiment of my invention.

FIG. 2 is a side view of a first embodiment of my invention.

FIG. 3 is a front view of a first embodiment of my invention as it would point toward the skin surface being inspected.

FIG. 4 is a top view of a first embodiment of my invention.

FIG. 5 is an operational view of a way a first embodiment of my invention can be used.

FIG. 6 is a second operational view of a way a first embodiment of my invention can be used.

### Drawings-Reference Numerals

10 webcam

20 handle

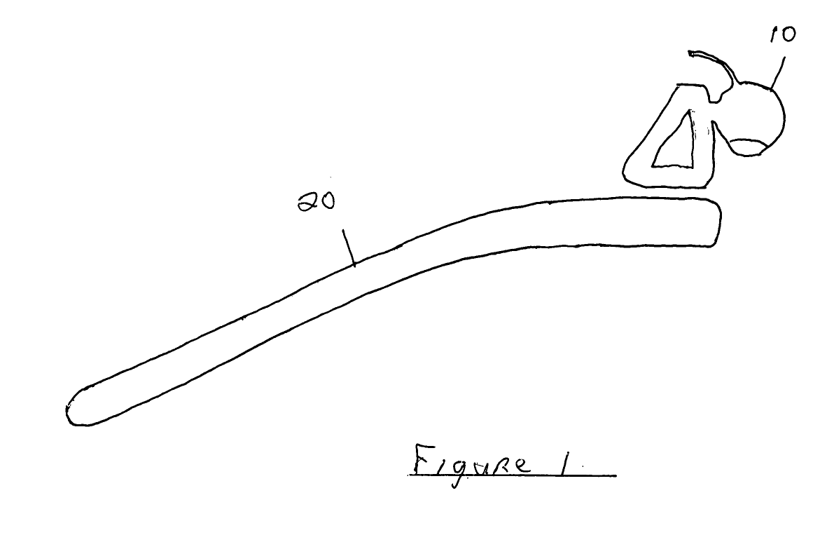

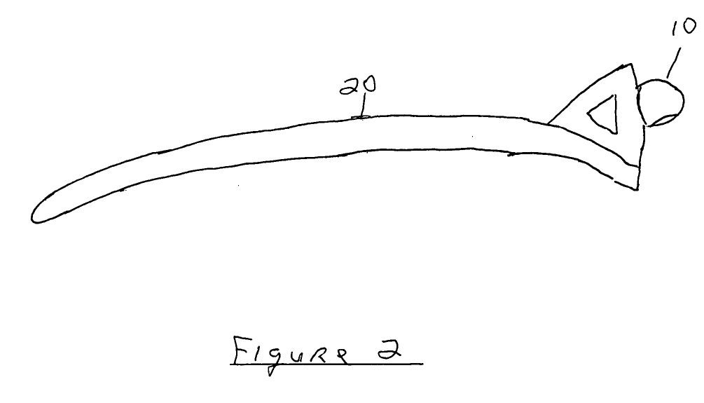

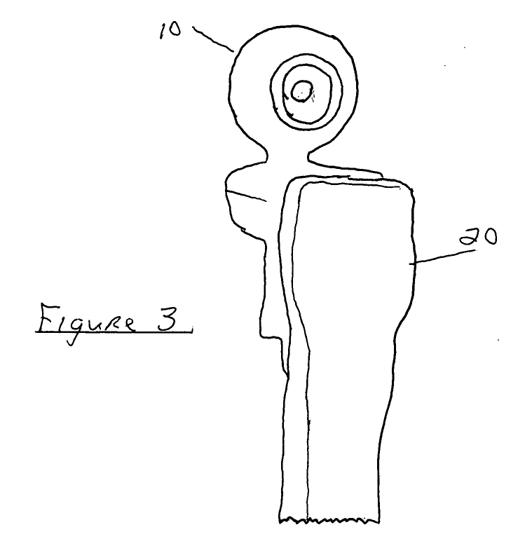

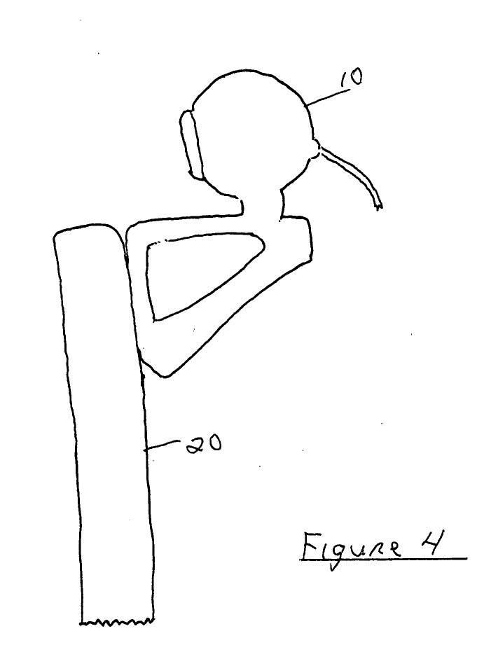

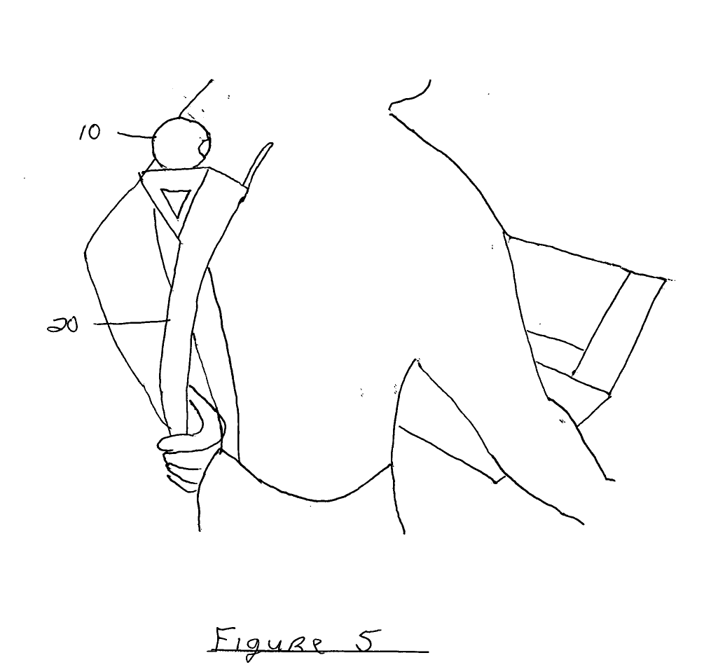

### DETAILED DESCRIPTION-FIGS. 1 AND 2-FIRST EMBODIMENT

One embodiment of the Back-Aid device is illustrated in FIG. 1 and FIG. 2 both in side views. The back aid device has handle 10 consisting of a rigid lightweight material with a curve midway of its length and a curve at its end. Connected to the outer curve at the handles end is a computer webcam 20. The webcam 20 is mounted by way of glue so that the webcam lens and line of sight is pointed toward the end of the handles inward curve for an unobstructed view. The handle 10 curve in the middle allows clearance for the curvature of the human spine for close contact with the skin aiding for manipulation and positioning the webcam. As improved webcams increase the megapixel power, this will improve the images obtainable. This is a factor which is important to Dermatologists looking at images of skin abnormalities. Currently webcams are available in a megapixel power up to 10 and digital cameras are currently available up to 14.2 megapixels.

Digital cameras that could be connected to a computer for control through a computer could serve the same function as the Webcam in the Back-Aid device I have invented. In other embodiments there may be digital and video cameras in place of the webcam with controls connected to the computer for taking pictures and video similar to how a computer webcam is able to do.

### OPERATION-FIGS. 2,4,5,6

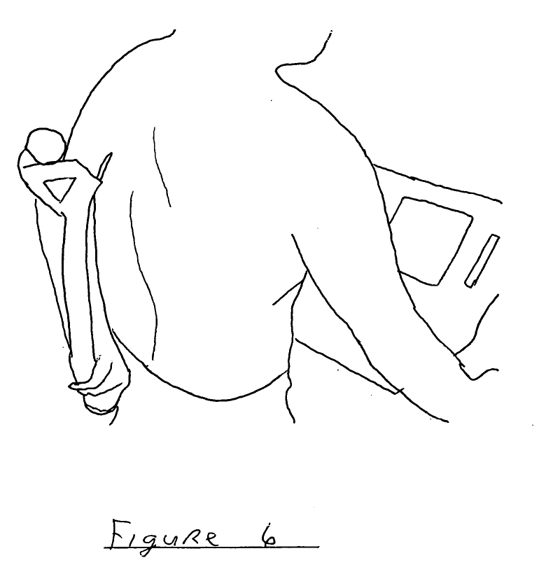

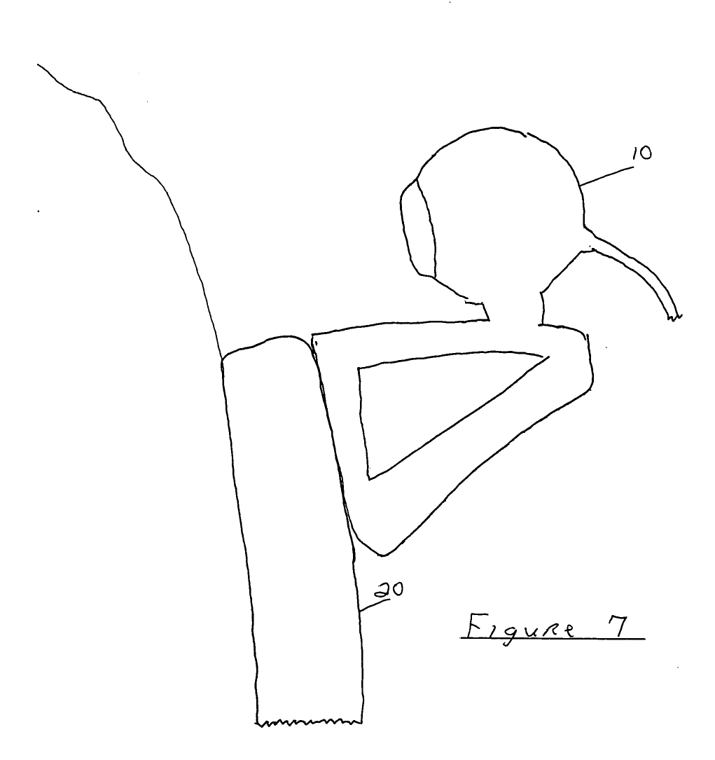

The manner of using the Back-Aid skin inspection device shown in FIG. 5 has the operator griping the handle as he/she is watches the display of the backside. The operator manipulates said device by arm movement with the webcam lens facing the skin surface. Once a specific area of skin is chosen to be imaged the said operator can activate a webcam picture with free hand by way of the computer due to the connection between the webcam and the computer. The field of view of webcam is demonstrated in FIGS. 2,4,5,6, as dotted lines emanating from the webcam lens. Pictures and video can then be taken by webcam and attached to e-mail and instantly sent to a doctor for determination of followup.

Alternative Embodiments would simply be replacing the computer webcam with miniaturized digital and video camera's which would be connected and controlled at the computer. With computer wireless technology people living in remote areas of the world can send immediate images to their chosen doctor for opinion if followup is needed.

### CONCLUSION, RAMIFICATIONS, AND SCOPE

Accordingly, the reader will see that the Back Aid device of the various embodiments can be used to inspect skin surfaces of the human backside. Webcams come in several different designs and megapixel strengths. The heart of one embodiment presented here is that the webcams being within three inches of the skin surface being inspected. The webcams can also be placed closer perhaps within an inch of skin surface for very close up imaging. Additionally, Infra-red light is being utilized to aid in skin cancer detection in part because of its ability to penetrate deeper into skin surfaces. The exact combination of different light of the light spectrum such as Infra Red may increase the effectiveness of this device. Miniaturized digital cameras and video cameras with controls wired or wireless to computers with longer or shorter positioning handles, staffs and even frames are envisioned.
Furthermore the Back Aid device has the additional advantage of ever increasing photos quality imaging as the megapixel strength increases with webcams, digital and video cameras. Although the description above contains different specifities these should not be construed as limiting the scope of the embodiments but as merely providing description of different embodiments which would result in the same device capabilities.

Thus the scope of the embodiments should be determined by the appended claims and their legal equivalents, rather then by the examples given.

Abstract: One embodiment of a back aid device would be the handle 20) connected to the computer webcam (10) with adhesive or small screws. The handle is made of a strong lightweight and rigid material to make manipulation of the device easy. Webcams (10) are of different designs and megapixel strengths and can be adapted to fit handle (10). Te greater the megapixel strength of the webcam the more detailed the resulting photo's. Other embodiments are described and shown.

What is claimed is:

1. A hand held self inspection device used as a means for visually inspecting skin of the human front or backside comprising:
a. A handle of rigid material approximately 20 inches in length and one inch in diameter with a curve in its main body and curved at its end
b. A computer webcam disposed and attached to said handle at the outer side of curved end whereby images of the body can be obtained by operator

2. Said computer webcam of claim 1 wherein is connected by wire or wireless to a computer whereby real time video can be seen on computer display

3. Said handle of claim 1 further including has a nylon flap with a velcro end for connecting complexion, sunblock and medicine application tools to said handle.

4. Said inspection device handle of claim 1 is made of plastic and said webcam joined to said handle by glue or screws

5. Said inspection handle of claim 1 has an infra red light emitting diode mounted near said webcam so as to emit light to direction of said webcams visual field

6. A new use of a computer webcam for self inspection of the human front and backside comprising:
a. an approximately 20 inch curved staff curved in the middle of its length and having a curved tip at its end
b. a computer web cam mounted to said curved staff at the outside surface of the curved end of said staff.

7. Said inspection device of claim 8 wherein allows pictures taken to be magnified by web cam software program for close up analysis of images.

8. Said inspection device of claim 1 wherein allows for substantially improved visual display and inspection of the human backside.

9. Said device of claim 10 wherein provides a method of skin inspection which can further include a digital or video camera attached to said staff with camera controls being connected to computer in place of said webcam.

10. Operator of said webcam of claim 8 wherein moves webcam human skin surfaces the webcam is facing within three inches of skin to look for abnormalities.

11. Said staff of claim 8 at the curved end across of mounted webcam rests against skin being inspected providing stability for smooth imaging by said webcam.

12. Said device of claim 10 webcam images obtained can be displayed on computer and compared to American Academy of Dermatology Skin Cancer Brochure images.

13. Said skin inspection device of claim 10 provides a means for self visual inspection of the back whereby early detection of skin cancers such as melanomas are critical for survival of such disease.

14. Said skin inspection device is superior to hand held mirrors for backside skin inspection due to no need of trying to look backwards into mirrors reflection of backside human skin.

15. A method for backside human skin self inspection by placing a computer webcam within three inches of said skin for close up imaging and immediate visual display from the combination of a specially designed rod of rigid material, a computer webcam and a computer of the operators choice.

16. Operator of claim 15 moves device over an area of skin and with free hand activates the picture recording function of said webcam by the computer allowing for pictures and video taken to be stored onto computer hard drive said device is connected to.

17. Operator of said device of claim 15 moves webcam to obtain different angles and photo's of suspected skin abnormalities

18. Images obtained of claim 15 are adjusted and magnified for study by operator

19. Operator of said device of claim 15 can add different spectrum's of light at different intensities such as infrared or white light to enhance images obtained.

20. Operator of claim 15 can send images obtained to a doctor
